(19) United States
(12) Patent Application Publication
(10) Pub. No.: US 2018/0064655 A1
(43) Pub. Date: Mar. 8, 2018

(54) Hydroxynorketamine Transdermal Patch

(71) Applicant: Steven Sounyoung Yu, Fairfax, VA (US)

(72) Inventor: Steven Sounyoung Yu, Fairfax, VA (US)

(21) Appl. No.: 15/697,420

(22) Filed: Sep. 6, 2017

Related U.S. Application Data
(60) Provisional application No. 62/384,126, filed on Sep. 6, 2016.

(51) Int. Cl. A61K 9/70; A61K 31/137

(52) U.S. Cl. CPC A61K 9/7084; A61K 31/137

(57) ABSTRACT

A transdermal patch comprising (2R,6R)-hydroxynorketamine or a conjugate thereof. The active agent may be in its free base form. The transdermal patch may comprise a drug-containing layer comprising the active agent and a carrier material. The transdermal patch may comprise an adhesive. Examples of adhesive materials that could be used include pressure-sensitive acrylic adhesives, pressure-sensitive silicone adhesives, or other type of pressure-sensitive adhesive.

### TECHNICAL FIELD

This invention relates to a transdermal patch for delivering an active agent to a patient.

### BACKGROUND

Ketamine has demonstrated rapid and robust efficacy as an antidepressant. Remarkably, antidepressant effects are observed within hours following a single administration, and persist for approximately one week. Although discovery of ketamine's antidepressant efficacy has elicited tremendous excitement in the field, its clinical use is limited because of its abuse potential and sensory-dissociation side effects.

However, it was recently shown that the (2R,6R)-hydroxynorketamine (HNK) metabolite of ketamine is responsible for the antidepressant effect observed with ketamine treatment. See Panos Zanos et al, “NMDAR inhibition-independent antidepressant actions of ketamine metabolites” (26 May 2016) Nature, 533:481-486. Importantly, (2R,6R)-HNK lacks ketamine-related side-effects. These discoveries have relevance for the development of next generation, rapid-acting antidepressants.

### SUMMARY

The active agent of this invention is (2R,6R)-HNK or a conjugate thereof. In some embodiments, the active agent is in its free base form. One aspect of my invention is a transdermal drug-delivery patch. The transdermal patch comprises a drug-containing layer comprising the active agent and a carrier material. The active agent may be provided in a relatively purer form relative to other stereoisomers or its enantiomer. The patch or the drug-containing layer may contain any suitable amount of the active agent. The active agent may be delivered transdermally to the patient at any suitable rate.

In some embodiments, the carrier material comprises an adhesive. The adhesive may comprise polymers having relatively few or no carboxyl functional groups, such as low carboxyl content polymers. The adhesive may exclude or limit the amount of materials that are not low carboxyl content polymers.

The adhesive may be an acrylic adhesive comprising acrylic polymers having relatively few or no carboxyl functional groups, such as low carboxyl content acrylic polymers. The drug-containing layer may comprise an acrylic adhesive, but exclude or limit the amount of acrylic polymers that are not low carboxyl content acrylic polymers.

The adhesive may be a silicone adhesive. The silicone adhesive may be amine-compatible. In some embodiments, the carrier material comprises both a silicone pressure-sensitive adhesive and an acrylic pressure-sensitive adhesive. In some cases, the silicone pressure-sensitive adhesive is amine-compatible and the acrylic adhesive comprises an acrylic polymer having relatively few or no carboxyl groups. In some embodiments, the carrier material comprises a first pressure-sensitive adhesive that is an acrylic adhesive and a second pressure-sensitive adhesive that is a silicone adhesive.

The drug-containing layer may have a low water content. The drug-containing layer may exclude or limit the amount of acidic compounds. The drug-containing layer may contain ammonia or a compound having a primary, secondary, or tertiary amine group. The drug-containing layer may comprise an anti-nucleating agent.

The transdermal patch may have any of the aforementioned elements or parameters in any combination. In some embodiments, the transdermal patch comprises a backing layer, a release liner, and a drug-containing layer between the backing layer and the release liner.

Another aspect of my invention is a transdermal patch product. The product comprises a sealed packaging with a transdermal patch of my invention within the sealed packaging. The packaging may be vacuum sealed.

Another aspect of my invention is a method of using any of the transdermal patches described herein. In some embodiments, the method is for delivering the active agent to a patient. In some embodiments, the method is for treating a psychiatric disorder in a patient. In some embodiments, the method comprises applying the transdermal patch onto the patient's skin.

Another aspect of my invention is a method of making any of the transdermal patches described herein. In some embodiments, the method comprises making a drug mixture of the active agent, an adhesive, and a solvent liquid. The solvent liquid may comprise an organic solvent and have a low water content.

The drug mixture is applied onto a foundation as a flat layer. In some embodiments, the drug mixture is dried until a drug-containing layer having a low water content is formed. In some embodiments, the method further comprises laying a release liner or backing layer on or over the drug-containing layer.

In some embodiments, the method comprises making a drug mixture of the active agent and a heat-melted pressure-sensitive adhesive. The method further comprises applying the drug mixture onto a foundation as a flat layer.

### BRIEF DESCRIPTION OF THE DRAWINGS

FIG. 1 shows an example of a lysine conjugate of (2R,6R)-HNK.

FIG. 2 show examples of ester conjugates of (2R,6R)-HNK.

FIG. 3 shows a polydimethylsiloxane polymer (PDMS) cross-linked with a siloxane resin to form a networked silicone polymer structure.

FIG. 4 shows an example of a single layer, drug-in-adhesive type patch in cross-section side view.

FIGS. 5A and 5B show an example of a transdermal patch having a drug matrix-diffusion configuration.

### DETAILED DESCRIPTION

### 1. Hydroxynorketamine

The transdermal patch comprises an active agent, which is (2R,6R)-HNK or a conjugate thereof. The active agent may be provided in any suitable pharmaceutically-acceptable form. In some embodiments, the active agent is in its free base form. In this instance, the active agent would be in a non-salt form (as opposed to, for example, being provided in the hydrochloride salt form in which the amine would be protonated as an ammonium ion). In some embodiments, the active agent is in a salt form, such as a hydrochloride salt.

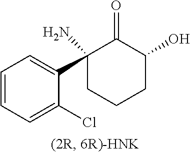 [SMILES: N[C@@]1(c2ccccc2Cl)CCC[C@@H](O)C1=O]

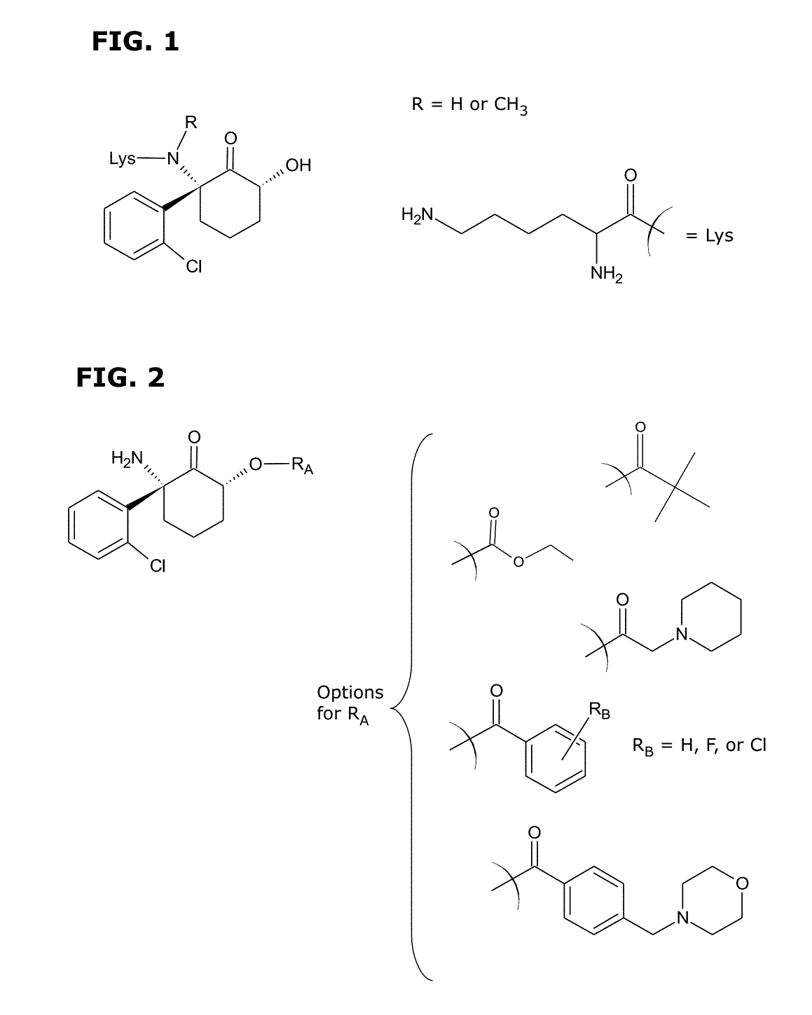

Conjugated forms of (2R,6R)-HNK are also possible. In some embodiments, the active agent is an amide conjugate (such as lysine or arginine conjugate). In some embodiments, the active agent is an ester conjugate. Amide and ester conjugates of (2R,6R)-HNK are described in U.S. Patent Application Publication No. 2014/0296241 (Irving Wainer et al), which is incorporated by reference herein. An example of a lysine conjugate of (2R,6R)-HNK is shown in FIG. 1. Examples of ester conjugates of (2R,6R)-HNK are shown in FIG. 2.

(2R,6R)-HNK free base may be synthesized and purified as described in Panos 2016, supra. Synthesis and purification of the hydrochloride salt form of (2R,6R)-HNK is also described in Panos 2016, supra.

An amine-containing drug can be provided with the amine group in ionized form by combining it with an acid. The most common acids used to ionize the amine group are hydrochloric acid and sulfuric acid. In product literature, the acid is sometimes shown separately without having reacted with the amine group (e.g. as “drug•HCl”).

For an amine-containing drug, there are many advantages to providing the drug with the amine in its ionized form, e.g. as an ammonium ion. Foremost, the ionized form of the drug is more water-soluble. This is especially important for injected or intravenously administered drugs. Second, the ionized salt form of the drug generally exists in a powdery solid form, which facilitates handling and processing. In contrast, the free amine form of drugs often occurs as an oily liquid or crystalline solid. Other potential advantages of the salt form of a drug are better thermal stability, better hydrolytic stability, better resistance to oxidation, better photostability, reduced hygroscopicity, or easier purification, handling, or processing.

In fact, there are many disadvantages to providing an amine-containing drug in its free base form. In its free base form, the drug is also more susceptible to oxidation. In its free base form, the drug is often less water-soluble. However, in a transdermal patch formulation, the free base form of a drug may have better skin penetration compared to the ionized salt form.

### 2. Purity

Hydroxynorketamine exists in different stereoisomers, and likewise, for its amide and ester conjugates. Whereas the active agent is (2R,6R)-HNK, or amide or ester conjugates thereof, other possible stereoisomers of hydroxynorketamine are (2S,6S)-HNK, (2R,6S)-HNK, and (2S,6R)-HNK (or amide or ester conjugates thereof). Note that (2R,6R)-HNK and (2S,6S)-HNK are enantiomers (and likewise, their corresponding amide or ester conjugates). The active agent may be provided in a mixture with other stereoisomer(s) of hydroxynorketamine. For example, the active agent (2R,6R)-HNK can be provided in a racemic mixture with (2S,6S)-HNK.

Providing the active agent in a more pure stereoisomer form may be beneficial for improving drug loading through the skin (instead of having to compete with other isomer forms). In some embodiments, the drug-containing layer or the transdermal patch contains less than 15 wt % of any other stereoisomer of the active agent, relative to the amount of the active agent (in the drug-containing layer or the transdermal patch); in some cases, less than 10 wt %; and in some cases, less than 6 wt %. In some embodiments, the drug-containing layer or the transdermal patch substantially excludes any other stereoisomers of the active agent. As used herein, the term “substantially excludes” with respect to an ingredient means that the ingredient is present in only incidental amounts as impurities or contaminants.

In some embodiments, the drug-containing layer or the transdermal patch contains less than 15 wt % of the opposite enantiomer of the active agent relative to the amount of the active agent (in the drug-containing layer or the transdermal patch); in some cases, less than 10 wt %; and in some cases, less than 6 wt %. In some embodiments, the drug-containing layer or the transdermal patch substantially excludes the opposite enantiomer of the active agent.

### 3. Dosage Amount

The transdermal patch or the drug-containing layer may contain any suitable amount of the active agent. One patch or multiple (two or more) patches may be used to deliver a therapeutically-effective amount of the active agent. In some embodiments, the patch or the drug-containing layer contains 10-250 mg of the active agent; in some cases, 20-180 mg; and in some cases, 20-90 mg.

It is possible that the active agent is contained in multiple layers of the transdermal patch. In some embodiments, the patch contains 10-250 mg of the active agent; in some cases, 20-180 mg; and in some cases, 20-90 mg.

Compared to an intravenous administration, the transdermal formulation may have incomplete bioavailability. Thus, a relatively higher amount of the active agent may be used as compared to intravenous administration. In some embodiments, the drug-containing layer or the patch contains at least 50 mg of the active agent; and in some cases, in the range of 50-180 mg of the active agent.

On the other hand, increasing the purity of the active agent relative to other non-active metabolites of ketamine or non-active stereoisomers of hydroxynorketamine may allow a reduction in the dosage amount that is needed (e.g. as compared to a racemic mixture). In some embodiments, the drug-containing layer or the patch contains 75 mg or less of the active agent; and in some cases, in the range of 10-75 mg of the active agent.

In some embodiments, the drug-containing layer or transdermal patch contains 50 mg or less of the active agent; and in some cases, in the range of 10-50 mg. In some embodiments, the drug-containing layer or transdermal patch contains 40 mg or less of the active agent; and in some cases, in the range of 10-40 mg.

In some embodiments, the drug-containing layer or transdermal patch contains 35 mg or less of the active agent; and in some cases, in the range of 5-35 mg. In some embodiments, the drug-containing layer or transdermal patch contains 30 mg or less of the active agent; and in some cases, in the range of 5-30 mg. In some embodiments, the drug-containing layer or transdermal patch contains 25 mg or less of the active agent; and in some cases, in the range of 5-25 mg.

The active agent in the patch may be distributed in the patch in any suitable manner. Considering the patch as a whole, a specified amount of the active agent may be contained in a single drug-containing layer or multiple (two or more) drug-containing layers. That is, when referring to the amounts contained in the patch as a whole, the above-specified amounts of the active agent may be contained in the drug-containing layer and in optional additional one or more drug-containing layers of the patch. Accordingly, the specified amounts being in the drug-containing layer or the patch can be alternate embodiments. That is, one embodiment may be the specified amount as being the amount of the active agent in the drug-containing layer; whereas another embodiment may be the specified amount as being the total amount of the active agent in the patch as a whole.

### 4. Dose Delivery

The patch may be designed to deliver the active agent to the patient at any suitable rate. As demonstrated in U.S. Pat. No. 6,348,211 (Juan Mantelle et al), among others, the desired delivery rate can be achieved by varying the selection of ingredients, the amount of the ingredients, how the formulation is made, and other formulation process parameters. In some embodiments, the transdermal patch delivers at least 5 mg of the active agent transdermally to the patient over the initial 24 hours after applying the patch; in some cases, 5-175 mg; and in some cases, 5-120 mg.

### 5. Drug-Containing Layer and Carrier Material

The transdermal patch comprises a drug-containing layer, which comprises the active agent. In addition to the active agent, the drug-containing layer comprises a carrier material for carrying the active agent. The active agent is mixed with the carrier material (e.g. blended, dispersed therein, encapsulated therein, etc.). In general, the carrier material comprises ingredients that are suitable for transdermal drug delivery, such as adhesives, solvents, additives, adjuvants, plasticizers, tackifiers, skin penetration enhancers, crosslinking agents, or other excipient substances. Such materials are described in Heather Benson & Adam Watkinson (eds.), Topical and Transdermal Drug Delivery: Principles and Practice (2012) and Richard Guy & Jonathan Hadgraft (eds.), Transdermal Drug Delivery, 2nd edition (2003); which are incorporated by reference herein.

The active agent can be admixed in the carrier material in any suitable way, including homogeneous admixtures, heterogeneous admixtures, or a combination thereof. For example, the drug may be homogenously dispersed in the drug-containing layer. In another example, the drug may form microspheres within a crosslinked polymer matrix of the drug-containing layer.

### 6. Water Content

The drug-containing layer may have a low water content. This may be beneficial for helping to avoid ionization of the amine group on the active agent. In some embodiments, the drug-containing layer contains less than 8% water (as liquid or vapor) by weight of the formulation; in some cases, less than 5%; and in some cases, less than 3%. In some embodiments, the drug-containing layer is non-aqueous (meaning that it substantially excludes water). This may be achieved in a variety of different ways, such as making the drug-containing layer without the use of an aqueous solvent or by evaporating the water from the drug-containing layer.

For purposes of definition herein, the water content of the drug-containing layer is measured after final drying of the product in the manufacturing process. The water content can be measured using the procedure given in United States Pharmacopeia (USP) 731 “Loss on Drying.” In this test, the drug-containing layer is weighed and stored in a desiccator chamber containing CaCl₂ desiccant at room temperature. Repeat weighing of the film at subsequent time intervals until it has a constant weight (dry to constant weight). The percent moisture content (using the wet basis formula) is the difference between initial weight and final weight, with respect to initial weight; (A−B)/A×100, where A is the initial weight of the sample and B is the final weight of the sample.

Preferably, this test should be conducted on the drug-containing layer(s) in isolation. However, for the sake of convenience, this test could be conducted with other components of the transdermal patch intact. For example, if the release liner serves as the foundation for making the drug-containing layer, then the test could be conducted with the release liner intact, but without the backing layer. In another example, if the backing layer serves as the foundation for making the drug-containing layer, then the test could be conducted with the backing layer intact, but without the release liner. It is also understood that for whatever water content X % that is the indicated result for the entire assembly or subassembly, the water content of the drug-containing layer(s) cannot be more than X %.

It is understood that this drying test will measure the amount of volatile solvents of any kind (not limited to water only). Nevertheless, it is also understood that for whatever moisture content X % that is the indicated result, the amount of water cannot be more than X %. For example, in a hypothetical situation where the drug-containing layer is made using a solvent mixture of methanol and water that are subsequently evaporated, and the moisture content test indicates 4 wt % moisture, then the water content must be 4 wt % or less (i.e. at most 4 wt %). For example, the actual moisture content may be 1 wt % methanol and 3 wt % water.

If more accurate measurements for the content of water alone are needed, the procedures given in United States Pharmacopeia (USP) 921 “Water Determination,” Method 1 (titration) or Method 2 (azeotropic toluene distillation) could be used instead.

### 7. Adhesive

In some embodiments, the carrier material comprises one or more skin-adhering adhesives. Any suitable amount of the adhesive(s) may be used in the drug-containing layer. In some embodiments, the one or more adhesives (combined total) constitute 40-98% of the drug-containing layer by weight. The adhesive may be admixed with the active agent in any suitable way, such as a homogeneous mixture, heterogeneous mixture, or a combination thereof. In some embodiments, the drug-containing layer comprises one or more pressure-sensitive adhesives.

The pressure-sensitive adhesive used in the drug-containing layer may comprise polymers having relatively few or no carboxyl (—COOH) functional groups. In some embodiments, the adhesive comprises a low carboxyl content polymer. As used herein, the term “low carboxyl content polymer” means a polymer in which less than 5 wt % of its monomers (with respect to the total weight of the polymer) have free carboxyl functional groups. In some cases, the low carboxyl content polymer has less than 1 wt % of its monomers (with respect to the total weight of the polymer) with free carboxyl functional groups.

The adhesive used in the drug-containing layer may exclude or limit the amount of materials that are not low carboxyl content polymers. In some embodiments, for the adhesive used in the drug-containing layer, materials that are not low carboxyl content polymers constitute at most 15 wt % of the adhesive; in some cases, constitute at most 10 wt %; and in some cases, constitute at most 5 wt %. In some embodiments, the adhesive substantially excludes any polymers having free carboxyl functional groups.

Pressure-sensitive adhesive formulations that could be used include those described in Istvan Benedek & Mikhail Feldstein (ed.), Handbook of Pressure-Sensitive Adhesives and Products: Technology Of Pressure-Sensitive Adhesives and Products, CRC Press (2009); Istvan Benedek, Pressure-Sensitive Adhesives and Applications, 2nd ed., Marcel Dekker (2004); Zbigniew Czech et al, “Pressure-Sensitive Adhesives for Medical Applications” (November 2011) in Wide Spectra of Quality Control (Isin Akyar, ed.), DOI: 10.5772/23827; all the preceding references are incorporated by reference herein.

In some embodiments, the pressure-sensitive adhesive is an acrylic adhesive. Examples of acrylic adhesives are described in Benedek & Feldstein 2009, supra at Chapter 5, “Acrylic Adhesives.” The acrylic adhesive comprises an acrylic polymer. The acrylic adhesive may comprise one or more different types of acrylic polymers. In some embodiments, the acrylic adhesive comprises two or more different types of acrylic polymers.

As used herein, “acrylic polymer” means that the polymer comprises acrylic monomers. There are many different types of acrylic monomers that could be used, including acrylic acids, esters of acrylic acids (e.g. alkyl esters of acrylic acid such as methyl, butyl, ethyl, and 2-ethylhexyl acrylate), acrylamides (e.g. acrylamide, methyl acrylamide, dimethyl acrylamide, diacetone acrylamide, octylacrylamide, etc.), acrylonitriles (e.g. acrylonitrile or methacrylonitrile), etc.

The acrylic polymer can comprise different types of acrylic monomers (e.g. as copolymers, block polymers, etc.). The acrylic polymer may include non-acrylic monomers, such as vinyl acetate, vinyl propionate, vinylpyrrolidone, N-vinyl caprolactam, butadiene, 2-chloroethyl vinyl ether, ethylene, styrene, or carboxylic acids such as itaconic acid or fumaric acid. The acrylic polymer may or may not be elastomeric.

Examples of acrylic polymers that could be used are described in U.S. Pat. No. 9,205,061 (Richard Hamlin et al); U.S. Pat. No. 9,238,012 (Takao Hiraoka et al); U.S. Pat. No. 8,916,191 (David Kanios); U.S. Pat. No. 7,608,282 (Peter Altenschöpfer et al); U.S. Pat. No. 8,524,273 (Satoshi Amano et al); U.S. Pat. No. 5,494,680 (Timothy Peterson); and U.S. Patent Application Publications No. 2014/0052081 (Kuo-Hua Yang et al); and No. 2003/0026829 (Subramanian Venkatraman); the disclosures of which are all incorporated by reference herein.

Examples of acrylic monomers include acrylic acid, methyl acrylate, ethyl acrylate, propyl acrylate, butyl acrylate, hexyl acrylate, heptyl acrylate, ethylbutyl acrylate, amyl acrylate, octyl acrylate, nonyl acrylate, ethylhexyl acrylate, decyl acrylate, dodecyl acrylate, tridecyl acrylate, methoxyethyl acrylate, ethoxyethyl acrylate, dimethylaminoethyl acrylate, tert-butylaminoethyl acrylate, ethyloctyl acrylate, glycidyl acrylate, hydroxymethyl acrylate, hydroxyethyl acrylate, hydroxypropyl acrylate, hydroxybutyl acrylate, hydroxyamyl acrylate, hydroxyhexyl acrylate, dimethylaminoethyl acrylate, tert-butylaminoethyl acrylate, trimethylolpropane triacrylate, and their corresponding methacrylics.

Example methacrylics of these monomers include methacrylic acid, methyl methacrylate, ethyl methacrylate, propyl methacrylate, butyl methacrylate, hexyl methacrylate, heptyl methacrylate, ethylbutyl methacrylate, amyl methacrylate, octyl methacrylate, nonyl methacrylate, ethylhexyl methacrylate, decyl methacrylate, dodecyl methacrylate, tridecyl methacrylate, methoxyethyl methacrylate, ethoxyethyl methacrylate, dimethylaminoethyl methacrylate, tert-butylaminoethyl methacrylate, ethyloctyl methacrylate, glycidyl methacrylate, hydroxymethyl methacrylate, hydroxyethyl methacrylate, hydroxypropyl methacrylate, hydroxybutyl methacrylate, hydroxyamyl methacrylate, hydroxyhexyl methacrylate, dimethylaminoethyl methacrylate, and tert-butylaminoethyl methacrylate.

Examples of commercially-available pressure-sensitive acrylic adhesives that may be used include those in the DURO-TAK (Henkel Corp.) line of acrylate copolymer adhesive products identified by product numbers 87-900A, 87-9301, 87-4098, 387-2510, 87-2510, 387-2287, 87-2287, 87-4287, 387-2516, 87-2516, 87-2074, 87-235A, 387-2353, 87-2353, 87-2852, 387-2051, 87-2051, 387-2052, 87-2052, 387-2054, 87-2054, 87-2194, and 87-2196; and the GELVA (Henkel Corp.) line of acrylate copolymer adhesive products identified by product numbers GMS 3083, GMS 3253, GMS 9073, and GMS 788; and the EUDRAGIT line of polymer products, such as anionic copolymers of methacrylic acid and ethyl acrylate (identified by product numbers L 30 D-55 and L 100-55); anionic copolymers of methacrylic acid and methyl methacrylate (identified by product numbers L 100, L 12,5, S 100, S 12,5); anionic copolymers of methyl acrylate, methyl methacrylate, and methacrylic acid (identified by product number FS 30 D); cationic copolymers of dimethylaminoethyl methacrylate, butyl methacrylate, and methyl methacrylate (identified by product numbers E 100, E 12,5, and E PO); and copolymers of ethyl acrylate, methyl methacrylate, trimethylammonioethyl methacrylate chloride (identified by product numbers RL 100, RL PO, RL 30 D, RL 12,5, RS 100, RS PO, RS 30 D, and RS 12,5); and neutral copolymers of ethyl acrylate and methyl methacrylate (identified by product numbers NE 30 D, NE 40 D, and NM 30 D).

In some embodiments, the acrylic polymer comprises two or more different types of monomers, at least one of the monomers being an acrylic monomer. Examples of such polymers include copolymers of methacrylic acid and methyl methacrylate; copolymers of methyl acrylate, methyl methacrylate, and methacrylic acid; copolymers of ethylene and methyl acrylate; and copolymers of butyl acrylate and vinyl acetate.

### 7(a). Non-Functional Acrylics

In the presence of an acid, the amine group on the active agent may become protonated, causing the active agent to be in ionized form. Although the ionic form of the active agent would improve aqueous solubility, it would impede penetration through the skin.

The acrylic adhesive used in the drug-containing layer may comprise acrylic polymers having relatively few or no carboxyl (—COOH) functional groups. In some embodiments, the acrylic adhesive comprises a low carboxyl content acrylic polymer. As used herein, the term “low carboxyl content acrylic polymer” means an acrylic polymer in which less than 15 wt % of its monomers (with respect to the total weight of the polymer) have free carboxyl functional groups. In some cases, the low carboxyl content acrylic polymer has less than 9 wt % of its monomers (with respect to the total weight of the polymer) with free carboxyl functional groups. For example, the acrylic polymer may be a co-polymer of methacrylic acid monomers and methyl methacrylate monomers, but the methacrylic acid monomers constitute less than 9 wt % of the co-polymer. In some embodiments, the acrylic adhesive used in the drug-containing layer comprises an acrylic polymer that substantially excludes free carboxyl functional groups.

In some embodiments, the drug-containing layer comprises a pressure-sensitive acrylic adhesive that comprises an acrylic polymer, but limits or excludes those that are not a low carboxyl content acrylic polymer. In some embodiments, the drug-containing layer comprises one or more pressure-sensitive acrylic adhesives, but less than 15 wt % of the acrylic polymers in the acrylic adhesive(s) are not a low carboxyl content acrylic polymer, relative to the total weight of the acrylic polymers of the adhesive(s); in some cases, less than 10 wt %; and in some cases, less than 5 wt %. In some embodiments, the drug-containing layer substantially excludes acrylic polymers that are not low carboxyl content acrylic polymers.

In some embodiments, the drug-containing layer comprises a pressure-sensitive acrylic adhesive in which less than 15 wt % of the acrylic polymers therein (relative to the total weight of the acrylic polymers of the adhesive) are acrylic polymers having free carboxyl functional groups; in some cases, less than 10 wt %; and in some cases, less than 5 wt %. In some embodiments, the drug-containing layer substantially excludes any acrylic polymers having free carboxyl functional groups.

Pressure-sensitive acrylic adhesive products that limit or exclude free carboxyl functional groups may be marketed as being “non-functional.” Examples of acrylic monomers having no free carboxyl functional groups include methyl acrylate, ethyl acrylate, methyl methacrylate, and ethyl methacrylate.

Examples of such non-functional pressure-sensitive acrylic adhesives include DURO-TAK product numbers 87-900A, 87-9301, and 87-4098; and GELVA product numbers GMS 3083, GMS 3253. Other examples of such non-functional acrylic polymers are disclosed in U.S. Pat. No. 8,916,191 (David Kanios), which is incorporated by reference herein.

In some embodiments, the pressure-sensitive acrylic adhesive comprises an acrylic polymer having free hydroxyl functional groups (—OH), but not free carboxyl functional groups (—COOH). Examples of adhesive products having such types of polymers include DURO-TAK product numbers 387-2510, 87-2510, 387-2287, 87-2287, 87-4287, 387-2516, 87-2516, and GELVA product number GMS 788.

### 7(b). Silicone Pressure-Sensitive Adhesives

Other types of pressure-sensitive adhesives that could be used include silicone pressure-sensitive adhesives, such as those described in Benedek & Feldstein 2009, supra at Chapter 6, “Silicone Pressure-Sensitive Adhesives,” which is incorporated by reference herein. Silicone pressure-sensitive adhesives comprise silicone polymers (also commonly referred to as polysiloxanes or silicones, which may also be crosslinked), which comprise an inorganic silicon-oxygen backbone (—Si—O—Si—O—Si—O—) with organic side groups attached to the silicon atoms (e.g. methyl or phenyl). Examples of silicone-based pressure-sensitive adhesives that could be used include those described in U.S. Pat. No. 8,916,191 (David Kanios), which is incorporated by reference herein.

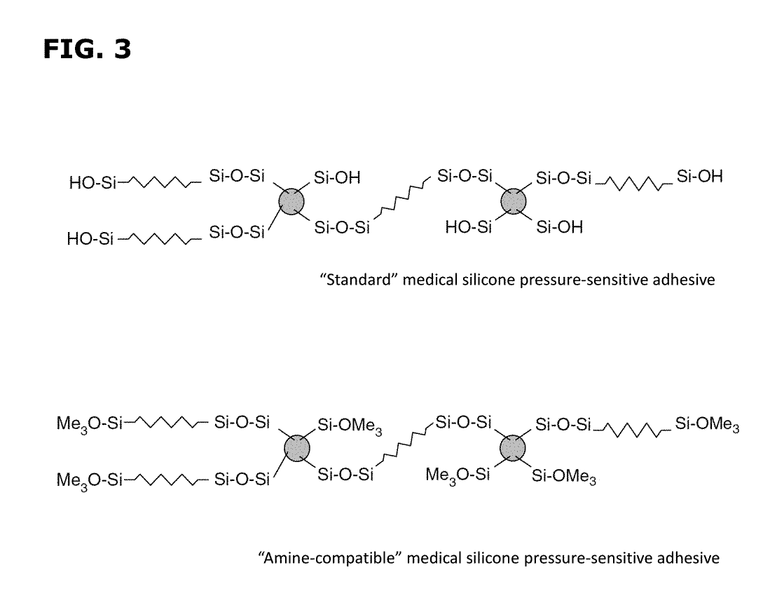

Many silicone pressure-sensitive adhesives are made by cross-linking silicone polymers with a siloxane resin via a condensation reaction to form a complex, three-dimensional silicone polymer network. See Benedek & Feldstein 2009, supra. FIG. 3 shows a polydimethylsiloxane polymer (PDMS) cross-linked with a siloxane resin to form a networked silicone polymer structure (taken from Benedek & Feldstein 2009, supra). In some embodiments, the silicone pressure-sensitive adhesive is made by a condensation reaction of a silicone polymer with a siloxane resin to form a networked silicone polymer structure.

Examples of commercially-available silicone pressure-sensitive adhesives that could be used include Dow Corning's line of standard silicone adhesive products identified by product numbers MD7-4502, MD7-4602, BIO-PSA 7-4401, BIO-PSA 7-4402, BIO-PSA 7-4501, BIO-PSA 7-4502, BIO-PSA 7-4601, BIO-PSA 7-4602, BIO-PSA 7-460X, BIO-PSA 7-450X, and BIO-PSA 7-440X. Examples of heat-meltable silicone pressure-sensitive adhesives include those in Dow Corning's line of silicone adhesive products identified by product numbers BIO-PSA 7-4560 and BIO-PSA Hot Melt Adhesive.

The free amine group on the active agent may undesirably act as a catalyst for the condensation of silicon-bonded hydroxyl groups in silicone pressure-sensitive adhesives. In some embodiments, the pressure-sensitive silicone adhesive is amine-compatible. The silicone adhesive product may be marketed as being “amine-compatible.” In some embodiments, the silicone pressure-sensitive adhesive comprises silicone polymers that are end-capped. As used herein, “end-capped” means that the polymer or resinous components have been chemically treated to reduce or eliminate the silicon-bonded hydroxyl content, preferably by substitution with a hydrocarbon radical such as a methyl group. Examples of end-capping are described in U.S. Pat. No. 6,337,086 (David Kanios et al) and U.S. Pat. No. RE 35,474 (John Woodard et al), which are both incorporated by reference herein. The term “end-capped” with respect to the silicone polymers does not necessarily mean that all the silanol groups are end-capped. It is possible that only some of the silanol groups on the silicone polymers are end-capped, while others are not. In some embodiments, the drug-containing layer comprises a pressure-sensitive silicone adhesive, but substantially excludes silicone polymers that are not end-capped.

In some cases, the amine-compatible pressure-sensitive silicone adhesive has a low silanol content. The product as marketed may be designated as having a low silanol content. See, for example, “Silicone Adhesives for Transdermal Drug Delivery Systems” (2015) Dow Corning Healthcare Solutions, Form No. 52-1070C-01. In some cases, the amount of free silanol groups in the silicone adhesive is less than 25,000 ppm; in some cases, less than 18,000 ppm; in some cases, less than 12,000 ppm; and in some cases, less than 9,000 ppm. The silanol content of silicones can be quantified by NMR or FTIR spectroscopy techniques, such as those described in U.S. Pat. No. 6,337,086 (David Kanios et al); André Colas, “Silicones in Pharmaceutical Applications” (2001) Dow Corning Corp.; and Greg Griffith, “Quantitation of silanol in silicones by FTIR spectroscopy” (1984) Ind Eng Chem Prod Res Dev. 23(4):590-593.

Examples of amine-compatible silicone polymers include those described in U.S. Pat. No. RE 35,474 (John Woodard et al) and U.S. Pat. No. 6,337,086 (David Kanios et al); André Colas, “Silicones in Pharmaceutical Applications” (2001) Dow Corning Corp.; Xavier Thomas, “Silicone Adhesives in Healthcare Applications” (2003) Dow Corning Healthcare Industry; Dow Corning's product literature “Dow Corning BIO-PSA: Amine-Compatible Silicone Adhesives” (2008) in Product Information: Healthcare. Examples of commercially available amine-compatible silicone pressure-sensitive adhesives include those in Dow Corning's line of amine-compatible silicone adhesive products identified by product numbers BIO-PSA 7-4101, BIO-PSA 7-4102, BIO-PSA 7-4201, BIO-PSA 7-4202, BIO-PSA 7-4301, BIO-PSA 7-4302, BIO-PSA 7-430X, BIO-PSA 7-420X, BIO-PSA 7-410X. As one example, the amine-compatible silicone pressure-sensitive adhesive may comprise networked silicone polymers in which the silanol groups are capped with trimethylsiloxy groups.

### 7(c). Other Pressure-Sensitive Adhesives

Other types of pressure-sensitive adhesives that could be used include rubber-based pressure-sensitive adhesives (also known as elastomeric adhesives), such as those described in Benedek & Feldstein 2009, supra at Chapter 2, “Rubber-Based Pressure-Sensitive Adhesives,” which is incorporated by reference herein. The rubber-based pressure-sensitive adhesive comprise rubber or elastomeric polymers. Examples of rubber or elastomeric polymers used in rubber-based pressure-sensitive adhesives include natural rubber polymers, polyisoprene polymers, polyisobutylene polymers, butyl rubber polymers (copolymers of isobutylene and isoprene), nitrile rubber polymers, and styrene-butadiene polymers.

Other types of pressure-sensitive adhesives that could be used include block copolymer-based pressure-sensitive adhesives, such as those described in Benedek & Feldstein 2009, supra at Chapter 3, “Block Copolymer-Based Hot-Melt Pressure-Sensitive Adhesives,” which is incorporated by reference herein. Examples of styrene block copolymers include styrene-isoprene-styrene (SIS), styrene-butadiene-styrene (SBS), styrene-isoprene/butadiene-styrene (SIBS), styrene-ethylene/butylene-styrene (SEBS), and styrene-ethylene/propylene-styrene (SEPS) block copolymers. Examples of commercially-available styrene block copolymers that could be used include those in the KRATON line of products.

Other types of pressure-sensitive adhesives that could be used include polyisobutene-based pressure-sensitive adhesives as described in Benedek & Feldstein 2009, supra at Chapter 4, “Polyisobutene-Based Pressure-Sensitive Adhesives,” which is incorporated by reference herein. Polyisobutene-based pressure-sensitive adhesives comprise vinyl polymers that comprise isobutylene monomers. A commercial example of a polyisobutene-based (also sometimes referred to as polyisobutylene) pressure-sensitive adhesive is DURO-TAK 87-6908.

Other types of pressure-sensitive adhesives that could be used include urethane-based pressure-sensitive adhesives, such as those described in Benedek & Feldstein 2009, supra at Chapter 11, “Pressure-Sensitive Adhesives Based on Polyurethanes,” which is incorporated by reference herein. Other types of pressure-sensitive adhesives that could be used include those containing polybutadiene, poly(ethylene-vinyl acetate) (PEVA; copolymer of ethylene and vinyl acetate), or polyvinyl ethers.

### 8. Acid/Base Substances

To maintain the active agent in its free base form, the drug-containing layer may limit or exclude compounds having acidity and capable of donating protons to the amine group on the active agent. Examples of such compounds include fatty acids (e.g. oleic acid, linoleic acid, valeric acid, lauric acid, etc.), 4-aminobenzoic acid, levulinic acid, etc.

In some embodiments, the drug-containing layer contains less than 15 wt % of any compound having a carboxylic acid functional group; in some cases, less than 7 wt %; and in some cases, less than 3 wt %. In some cases, the drug-containing layer substantially excludes any compounds having a carboxylic acid functional group.

To maintain the active agent in its free base form, the drug-containing layer may comprise compound(s) capable of withdrawing protons to prevent the amine group on the active agent from becoming protonated. In some embodiments, the drug-containing layer contains ammonia or a compound having a primary, secondary, or tertiary amine group. Examples of such amine-containing compounds include diethanolamine, triethanolamine, diethylamine, di-isopropanol amine, dimethylaminohexanoic acid dodecyl ester, dodecyl-N,N-dimethylamino acetate, p-phenylenediamine, and dodecyl 2-N,N-dimethylaminopropionate (DDAIP). Because amide groups contain an amine group therein, the term “amine-containing compound” also includes compounds that have amide group(s). Examples of such amine-containing compounds include urea and derivatives thereof (such as 1,3-diphenyl-urea), polyvinylpyrrolidone, dimethylacetamide, dimethylformamide, pyrrolidone and derivatives thereof, azone and derivatives thereof, etc.). In some embodiments, the amine-group compound has a molecular weight of less than 500 g/mol; in some cases, less than 300 g/mol. In some embodiments, the amount of ammonia or amine-group compound in the drug-containing layer is in the range of 0.5-4% by weight.

In some embodiments, the drug-containing layer comprises an organic or inorganic base as described in U.S. Pat. No. 6,673,363 (Luo et al), which is incorporated by reference herein. The optimum amount of the organic or inorganic base will depend on a variety of factors such as the strength of the base, the molecular weight of the base, the presence of acidic species in the drug-containing layer, etc.

Inorganic bases that could be used include inorganic hydroxides such as ammonium hydroxide, alkali metal hydroxides, and alkaline earth metal hydroxides. Examples of alkali metal hydroxides include sodium hydroxide and potassium hydroxide; examples of alkali earth metal hydroxides include calcium hydroxide and magnesium hydroxide. In some embodiments, the amount of inorganic hydroxide in the drug-containing layer is in the range of 0.5-4% by weight.

Inorganic bases also include inorganic oxides such as magnesium oxide, calcium oxide, and the like. In some embodiments, the amount of inorganic oxide in the drug-containing layer is in the range of 2-20% by weight.

Inorganic bases also include inorganic salts of weak acids such as ammonium phosphate (dibasic); alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic); alkaline earth metal salts of weak acids such as magnesium phosphate and calcium phosphate; and the like. In some embodiments, the amount of inorganic salt of weak acid in the drug-containing layer is in the range of 2-20% by weight.

Organic bases include compounds having an amino group, amido group, an oxime, a cyano group, an aromatic or non-aromatic nitrogen-containing heterocycle, a urea group, or combinations thereof. More specifically, examples of suitable organic bases are nitrogenous bases, which include primary amines, secondary amines, tertiary amines, amides, oximes, cyano (—CN) containing groups, aromatic and non-aromatic nitrogen-containing heterocycles, urea, and mixtures thereof. Examples of nitrogenous organic bases that could be used include 2-amino-2-methyl-1,3-propanediol, 2-amino-2-methyl-1-propanol, diethanolamine, triethanolamine, butylamine, dimethylamine, cyclohexylamine, ethylenediamine, isopentylamine, ethanolamine, phenethylamine, piperidine, pyrrolidine, and trimethylamine. In some embodiments, the organic base has a molecular weight of less than 500 g/mol; in some cases, less than 300 g/mol. In some embodiments, the amount of organic base in the drug-containing layer is in the range of 0.5-4% by weight.

### 9. Anti-Nucleating Agent

The free amine form of the active agent may be susceptible to crystallization (as compared to the ionized form), which would impede its penetration through skin. In some embodiments, the drug-containing layer comprises one or more anti-nucleating agents. As used herein, the term “anti-nucleating agent” refers to any material that serves to prevent crystallization of the active agent in the drug-containing layer. In some embodiments, the amount of anti-nucleating agent in the drug-containing layer is in the range of 0.5-25% by weight; and in some cases, 3-15% by weight. In some embodiments, the weight ratio of anti-nucleating agent relative to the active agent in the drug-containing layer is in the range of 0.005:1 (excess active agent) to 10:1 (excess of anti-nucleating agent); and in some cases, in the range of 0.01:1 to 5:1 weight ratio. In some embodiments, the molar ratio of anti-nucleating agent relative to the active agent in the drug-containing layer is in the range of 0.1:1 (excess active agent) to 50:1 (excess of anti-nucleating agent); and in some cases, in the range of 0.3:1 to 20:1 molar ratio. Anti-nucleating agents may be particularly useful in situations where the active agent is in a supersaturated condition in the drug-containing layer.

In some embodiments, the anti-nucleating agent is a polymer. In some cases, the polymer used as the anti-nucleating is different from the polymer used as an adhesive. In some cases, the anti-nucleating agent is a cellulose polymer, such as methyl cellulose, ethyl cellulose, hydroxyalkylcelluloses such as hydroxyethyl cellulose, hydroxypropyl cellulose (HPC), hydroxypropyl methylcellulose (HPMC), ethyl hydroxyethyl cellulose, hydroxypropyl methylcellulose phthalate, hydroxyethyl methylcellulose, and carboxylated cellulose derivatives such as carboxymethyl cellulose.

In some embodiments, the anti-nucleating agent is an acrylic polymer as described above. Other examples of polymeric anti-nucleating agents that could be used include poly(vinyl alcohol) (PVA) and polyethylene glycol (PEG, also known as polyethylene oxide PEO), and polyvinylpyrrolidone. The term “polyvinylpyrrolidone” means a polymer that comprises vinylpyrrolidone (also referred to as N-vinylpyrrolidone or N-vinyl-2-pyrrolidone) as a monomeric unit. The polyvinylpyrrolidone may have one or more different types of monomer units (e.g. homopolymers, copolymers, etc.). Examples of polyvinylpyrrolidone copolymers include vinylpyrrolidone/vinyl acetate (VP/PA) copolymers and vinylpyrrolidone/dimethylamino-ethylmethacrylate copolymers. The polyvinylpyrrolidone may or may not be cross-linked (e.g. crospovidone).

In some embodiments, the anti-nucleating agent is a cyclodextrin. Examples of cyclodextrins include α-cyclodextrins, β-cyclodextrins, and γ-cyclodextrins. Examples of ρ-cyclodextrins include hydroxypropyl-β-cyclodextrin, methyl β-cyclodextrin, and randomly methylated-β-cyclodextrins (RAMEB).

### 10. Structure

As used herein, the term “transdermal patch” means a patch that is applied to the skin (as opposed to, for example, being applied to mucosa such as the inside of the mouth or intranasally). The transdermal patch may have any suitable structural organization. Examples of different transdermal patch configurations that could be used are described in K H Ramteke et al, “Transdermal Drug Delivery System: A Review” (2012)J Adv Scient Res. 3(1):22-35; Richa Sachan et al, “Transdermal Drug Delivery System: A Review” (2013) International Journal of Research & Development in Pharmacy & Life Sciences, vol. 3(1):748-765; A. Arunachalam et al, “Transdermal Drug Delivery System: A Review” (2010) Current Pharma Research, vol. 1(1); Dipen Patel et al, “Transdermal Drug Delivery System: A Review” (2012) The Pharma Innovation Journal, vol. 1(4); the preceding references are incorporated by reference herein.

Examples of transdermal patch configurations that could be used include single drug-in-adhesive layer configuration, multilayer drug-in-adhesive configuration, reservoir compartment configuration, drug matrix-diffusion configuration, permeable membrane-controlled configuration, or microreservoir configuration. In some embodiments, the transdermal patch does not have a separate rate-controlling membrane. In some embodiments, the transdermal patch does not operate by electrical iontophoresis.

In some embodiments, the patch comprises a backing layer, a release liner, and a drug-containing layer between the backing layer and the release liner. The patch may have a single drug-containing layer or have multiple (two or more) drug-containing layers. The multiple drug-containing layers may have the same or different compositions.

### 10(a). Backing Layer

As conventional, the transdermal patch comprises a backing layer. Examples of backing layers (also sometimes referred to as backing laminates, backing films, etc.) that could be used in this invention are described in U.S. Pat. No. 9,168,232 (Naohisa Kawamura et al); U.S. Pat. No. 9,314,470 (Prashant Patel et al); U.S. Pat. No. 9,173,855 (Hiroko Udagawa et al); U.S. Pat. No. 9,205,061 (Richard Hamlin et al); and U.S. Patent Application Publications No. 2008/0254073 (Sang Cheol Chi); No. 2016/0113908 (Abhijit Deshmukh et al); No. 2016/0095926 (David Kanios); No. 2015/0250738 (Su II Yum et al); No. 2015/0246007 (Robert Gale et al); No. 2016/0051489 (Richard Hamlin et al); No. 2016/0030362 (Jun Liao et al); and No. 2015/0283097 (Kensuke Murata); which are all incorporated by reference herein. Materials suitable for use as backing layers are well-known and can comprise films of polyester, polyethylene, vinyl acetate resins, polyolefin, ethylene/vinyl acetate copolymers, polyvinyl chloride, polyurethane, metal foils (e.g. aluminum foil or aluminum-coatings), non-woven fabric, or cloth.

### 10(b). Release Liner

The transdermal patch may comprise a release liner on the opposite face of the patch relative to the backing layer. When present, the release liner is removed before applying to the skin. Materials suitable for use as release liners are well-known and include paper fabric, polyethylene, polyvinylchloride, polyester, metalized laminate, polypropylene, and polystyrene. The material for the release liner may be coated with silicone or fluoropolymer to facilitate detachment. The release liner may be provided as a single unitary sheet or as multiple sheets (e.g. as two sheets with one overlapping the other).

Examples of release liners (also sometimes referred to as protective liners) that could be used in this invention are described in U.S. Pat. No. 9,314,470 (Prashant Patel et al); U.S. Pat. No. 9,314,527 (Jeremy Cottrell et al.); U.S. Pat. No. 9,155,712 (David Kanios et al.); U.S. Pat. No. 9,205,061 (Richard. Hamlin et al); and U.S. Patent Application Publications No. 2016/0113908 (AM Deshmukh et al); No. 2016/0051489 (Richard Hamlin et al.); No. 2016/0095926 (David Kanios); No. 2016/0030362 (Jun Liao et al); No. 2015/0283097 (Kensuke Murata); and No. 2015/0246006 (Kristin Jackson et al); which are all incorporated by reference herein.

### 10(c). Examples

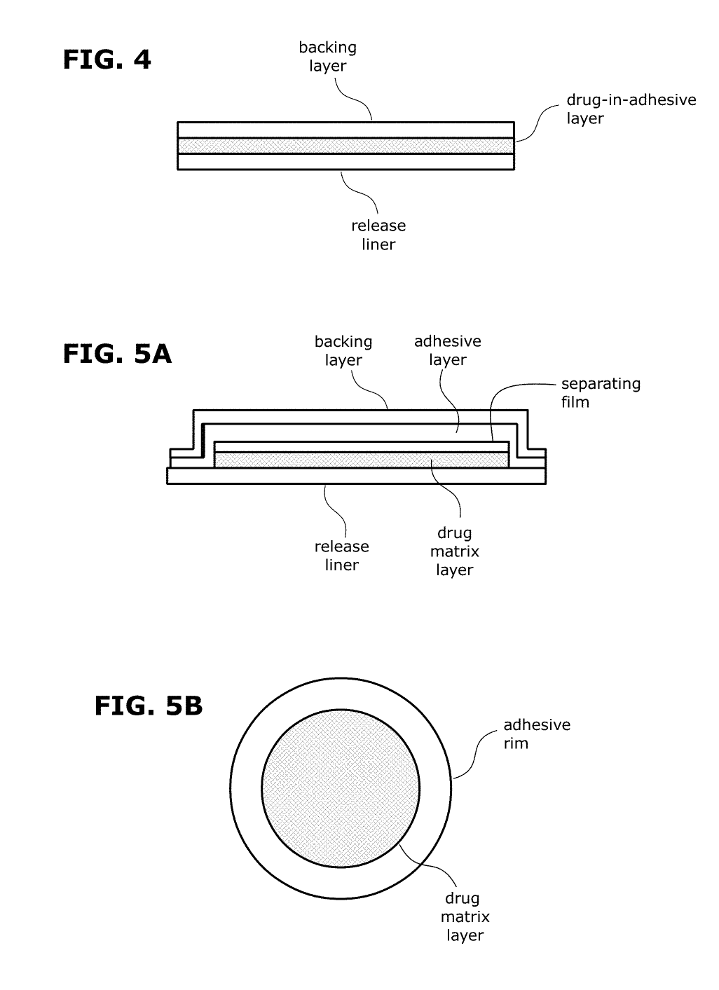

FIG. 4 shows an example of a single layer, drug-in-adhesive type patch in cross-section side view. The drug-in-adhesive layer contains a pressure-sensitive adhesive admixed with the active agent. A backing layer is laminated onto one face of the drug-in-adhesive layer and a release liner is laminated onto the other face.

FIGS. 5A and 5B show an example of a transdermal patch having a drug matrix-diffusion configuration. As shown in the cross-section side view of FIG. 5A, there is a drug-containing matrix layer holding the active agent within a polymer matrix of an adhesive material. Surrounding the drug-containing matrix disc is a pressure-sensitive adhesive layer. The pressure-sensitive adhesive layer is supported by a backing layer. There is a thin separating film between the drug matrix and the adhesive layer. The drug matrix is protected by a release liner. FIG. 5B shows a bottom view of the patch with the release liner peeled off. As seen in this view, the adhesive layer forms an adhesive rim around the drug matrix.

### 11. Packaging

The transdermal patch may be put into any suitable type of packaging. In some embodiments, the transdermal patch is contained in a sealed packaging. In its free amine form, the active agent may be more susceptible to oxidation (as compared to its ionized salt form). As such, in some cases, the packaging is vacuum sealed.

### 12. Method of Using

This invention also encompasses methods of using a transdermal patch for delivering the active agent to a patient or for treating a psychiatric disorder in a patient. Examples of psychiatric disorders that could be treated by the transdermal patch include depressive disorders as defined in the Diagnostic & Statistical Manual of Mental Disorders, 5th ed. (DSM 5), which is incorporated by reference herein. Depressive disorders include disruptive mood dysregulation disorder, major depressive disorder, persistent depressive disorder (dysthymia), premenstrual dysphoric disorder, substance/medication-induced depressive disorder, depressive disorder due to another medical condition, other specified depressive disorder, and unspecified depressive disorder; as well as other psychiatric conditions in which mood depression can be a primary symptom, such as bipolar disorder, post-traumatic stress disorder (PTSD), anxiety disorders (e.g. generalized anxiety disorder or social anxiety disorder), psychotic disorders such as schizoaffective disorder and schizophrenia, personality disorders, and eating disorders. These depressive disorders are often characterized by symptoms such as sad, empty, or irritable mood, diminished interest in activities, irritability, sleep problems, psychomotor slowing or agitation, changes in appetite, poor concentration or indecisiveness, excessive guilt or feelings of worthlessness, and suicidal ideations.

The method involves using any of the transdermal patches described herein. The patch may be applied by the patient him/herself or by someone else such as a companion, caretaker, spouse, parent, clinician, etc.

Repeated treatments with the transdermal patch is possible. In some embodiments, the method further comprises applying a fresh (second) transdermal patch within a time of one day to two weeks after applying the first transdermal patch; in some cases, within a time of two days to nine days; and in some cases, within a time of three to nine days. In some embodiments, a fresh patch is repeatedly applied at intervals of R days, wherein R is a value in the range of 1-14 (e.g. once daily, once every four days, once a week, etc.); in some cases, R is a value in the range of 2-9; and in some cases, R is a value in the range of 3-9.

### 13. Method of Making

Any suitable manufacturing technique could be used to make the transdermal patch.

### 13(a). Solvent Casting

The drug-containing layer could be made by a solvent-casting technique. In some embodiments, the method comprises making a drug mixture of the active agent, an adhesive, and a solvent liquid. The ingredients of the drug mixture may be combined in any suitable way. For example, the adhesive may be provided in a solvent liquid and the active agent could be mixed into the adhesive liquid. In another example, the ingredients may be provided separately and the adhesive is mixed into the solvent liquid and then the active agent is added to the mixture. Or in another example, the adhesive could be made by polymerization of monomers in a solvent liquid, and then the active agent could be added to the polymerized adhesive.

The method further comprises applying the drug mixture onto a foundation. In some embodiments, the foundation is provided by a backing layer, i.e. the backing layer serves as the foundation for applying the drug mixture. In some embodiments, the foundation is provided by a release liner, i.e. the release liner serves as the foundation for applying the drug mixture.

The method further comprises drying the solvent liquid to form a drug-containing layer. In some cases, the resulting drug-containing layer has a low water content as described above.

### 13(b). Solvent

The solvent liquid comprises one or more solvents. In some embodiments, the solvent liquid comprises an organic solvent and has a low water content. In some embodiments, less than 25% by weight of the solvent liquid is water; in some cases, less than 17%; in some cases, less than 10%; and in some cases, less than 5%. In some embodiments, the solvent liquid substantially excludes water (non-aqueous).

As used herein, “organic solvent” means a solvent having at least one carbon atom in its chemical formula. In some embodiments, the solvent liquid comprises one or more organic solvents that constitute at least 60% by weight of the solvent liquid; in some cases, at least 75%; and in some cases, at least 85%. In some cases, the solvent liquid consists substantially only of one or more organic solvents.

Examples of organic solvents include acetic acid, acetone, acetyl acetone, acetonitrile, benzene, 1-butanol, 2-butanol, 2-butanone, t-butyl alcohol, hexane, heptane, cyclohexane, cyclopentanone, carbon tetrachloride, chlorobenzene, chloroform, 1,2-dichloroethane, diethylene glycol, diethyl ether, bis(2-methoxyethyl) ether, dimethoxyethane, dimethyl formamide, dimethyl sulfoxide, dioxane, ethanol, ethyl acetate, ethylene glycol, glycerin, hexamethylphosphoramide (HMPA), hexamethylphosphorous triamide (HMPT), methanol, methylene chloride, N-methyl-2-pyrrolidinone, nitromethane, pentane, 1-propanol, 2-propanol, pyridine, tetrahydrofuran, benzene, toluene, benzaldehyde, N,N-dimethylaniline, triethyl amine, xylene, etc.

In some embodiments, the organic solvent is a polar organic solvent. This may be useful because the active agent may be more soluble in polar solvents than in non-polar solvents. The polar organic solvent may be protic or aprotic. Examples of polar aprotic organic solvents include N-methylpyrrolidone, tetrahydrofuran, ethyl acetate, acetone, dimethylformamide, acetonitrile, dimethyl sulfoxide, dichloromethane, dimethylformamide, and hexamethylphosphoric triamide. Examples of polar protic organic solvents include formic acid, n-propanol, n-butanol, t-butanol, isopropanol, nitromethane, ethanol, methanol, and acetic acid.

### 13(c). Heat-Melt

The drug-containing layer could also be made by a heat-melt technique. In some embodiments, the method comprises making a drug mixture of the active agent and a pressure-sensitive adhesive that is heat-melted. The ingredients of the drug mixture may be combined in any suitable way. For example, the pressure-sensitive adhesive may be melted first and then the active agent is added. Or alternatively, in another example, the pressure-sensitive adhesive may be mixed together with the active agent along with other ingredients, and then the mixture is heat-melted.

The method further comprises applying the drug mixture onto a foundation to form a drug-containing layer on the foundation. In some embodiments, the foundation is provided by a backing layer, i.e. the backing layer serves as the foundation for applying the drug mixture. In some embodiments, the foundation is provided by a release liner, i.e. the release liner serves as the foundation for applying the drug mixture.

This heat-melt technique can be a solvent-free approach to making the drug-containing layer. As a result, the drug-containing layer could be made with a low water content as described above.

### 13(d). Examples

Specific examples for manufacturing transdermal patches of the invention are given below. The order of steps, the amount of the ingredients, the reaction conditions (such as temperature, time durations, etc.), inclusion of other optional ingredients, and other process variables can be determined, modified, or adjusted through routine empirical exercises. The adhesive used to make the patch can be provided in any suitable form, such as a solid form (e.g. powder, granules, pellets, etc.) or mixed into an aqueous or organic solvent (e.g. as a solution, dispersion, or emulsion).

Experimental examples that could be used as guidance are given in U.S. Pat. No. 9,265,752 (Shuming Wang et al); U.S. Pat. No. 5,665,378 (Roosevelt Davis et al), U.S. Pat. No. 6,264,980 (Thomas Hille); U.S. Pat. No. RE 41,408 (Robert Reder et al); U.S. Pat. No. 6,660,295 (Tyler Watanabe et al); U.S. Pat. No. 6,953,590 (Yukino Owaki et al); U.S. Pat. No. 5,698,217 (Shari L. Wilking); U.S. Pat. No. 8,246,976 (Viet Nguyen); U.S. Pat. No. 8,632,802 (David Kanios); U.S. Pat. No. 9,283,174 (Tatsuya Mori et al); U.S. Pat. No. 6,555,129 (Christoph Arth et al); U.S. Pat. No. 5,861,170 (Thomas Kissel); U.S. Pat. No. 6,348,211 (Juan Mantelle et al); and U.S. Patent Application Publication No. 2014/0271792 (Jun Liao); which are all incorporated by reference herein.

### Example 1

Blend together acrylic pressure-sensitive adhesives DURO-TAK 87-2054, DURO-TAK 87-2051, and DURO-TAK 87-2516. Mix (2R,6R)-HNK free base into the adhesive blend. Use a film applicator tool to spread the wet blend onto a fluoropolymer-coated polyester film at a thickness of 0.5 mm. Alternatively, a spreading knife can be used for applying the wet blend. Dry the film to evaporate off the volatile solvents. Laminate with a polyethylene monolayer film as a backing layer. Cut the sheet into individual patches and seal inside black light-opaque pouches.

### Example 2

In an inert gas atmosphere, mix together 93 parts octyl acrylate and 7 parts acrylic acid in ethyl acetate solvent. To initiate polymerization, add a small amount of benzoyl peroxide while maintaining the temperature at 60° C. Add lysine conjugate of (2R,6R)-HNK free base, followed by addition of more ethyl acetate solvent. Coat a polyester release liner with this adhesive mixture to a thickness after drying of 20 μm. Dry the coating in an oven to give a drug-containing layer loaded with the active agent. Lay a 12 μm thick polyester backing onto the drug-containing adhesive layer.

### Example 3

Mix together isooctyl acrylate, acrylamide, and vinyl acetate (in a 75:5:20 ratio) into an organic solvent mixture of methanol and ethyl acetate (90:10 ratio). Add a small amount of benzoyl peroxide to initiate polymerization. Into this adhesive mixture, add (2R,6R)-HNK free base, glyceryl monolaurate, isopropyl myristate, and ethyl oleate. Spread the wet formulation onto a silicone-coated polyester release liner. Dry in an oven and then laminate the other side with a polyethylene backing film. Cut into individual patches and seal inside light-opaque metal foil pouches.

### Example 4

Mix together BIO-PSA 7-4602 (Dow Corning; silicone adhesive in ethyl acetate solvent) and DURO-TAK 87-6908 (Henkel; polyisobutylene). Mix an ester conjugate of (2R,6R)-HNK free base into the solvent/polymer mixture. Spread the wet blend onto a fluoropolymer-coated release liner. Dry at room temperature and then in an oven at 60° C. Attach a backing layer to the drug-adhesive layer.

### Example 5

Mix together 60 parts of a silicone adhesive (30 parts BIO-PSA 7-4602 and 30 parts BIO-PSA 7-4502), 20 parts of an acrylic adhesive (DURO-TAK 87-4194) and 20 parts of (2R,6R)-HNK free base until homogenous mixture is formed. Spread the wet blend onto a release liner. Dry in an oven to drive off the volatile solvents. Attach a backing layer to the drug-adhesive layer.

### Example 6

Mix together 30 wt % BIO-PSA 7-4403 silicone adhesive, 40 wt % DURO-TAK 87-2296 acrylic adhesive, and 30 wt % (2R,6R)-HNK free base until homogenous mixture is formed. Spread the wet blend onto a release liner. Dry in an oven to drive off the volatile solvents. Attach a backing layer to the drug-adhesive layer.

### Example 7

Mix together 56 wt % BIO-PSA 7-4403 silicone adhesive, 20 wt % DURO-TAK 87-2296 acrylic adhesive, 4 wt % oleyl alcohol, and 20 wt % (2R,6R)-HNK free base until homogenous mixture is formed. Spread the wet blend onto a release liner. Dry in an oven to drive off the volatile solvents. Attach a backing layer to the drug-adhesive layer.

### Example 8

Mix together 50 wt % BIO-PSA 7-4302 silicone adhesive, 40 wt % DURO-TAK 87-2296 acrylic adhesive, and 10 wt % (2R,6R)-HNK free base until homogenous mixture is formed. Spread the wet blend onto a release liner. Dry in an oven to drive off the volatile solvents. Attach a backing layer to the drug-adhesive layer.

### Example 9

Mix together 70 wt % BIO-PSA 7-4201 silicone adhesive, 10 wt % DURO-TAK 87-2296 acrylic adhesive, and 20 wt % (2R,6R)-HNK free base until homogenous mixture is formed. Spread the wet blend onto a release liner. Dry in an oven to drive off the volatile solvents. Attach a backing layer to the drug-adhesive layer.

### Example 10

Mix together 30 wt % BIO-PSA 7-4502 silicone adhesive, 30 wt % BIO-PSA 7-4602 silicone adhesive, 30 wt % DURO-TAK 87-4194 acrylic adhesive, and 10 wt % (2R,6R)-HNK free base until homogenous mixture is formed. Spread the wet blend onto a release liner. Dry in an oven to drive off the volatile solvents. Attach a backing layer to the drug-adhesive layer.

### Example 11

Mix together BIO-PSA 7-4302 silicone adhesive (amine-compatible), DURO-TAK 87-900A (non-functional acrylic adhesive), and (2R,6R)-HNK free base until homogenous mixture is formed. Spread the wet blend onto a release liner. Dry in an oven to drive off the volatile solvents. Attach a backing layer to the drug-adhesive layer.

### Example 12

Mix together BIO-PSA 7-4402 (Dow Corning; silicone adhesive in ethyl acetate solvent) and a non-elastomeric acrylic pressure-sensitive adhesive. Add oleyl alcohol, KOLLIDON 30 (BASF; polyvinylpyrrolidone), and (2R,6R)-HNK free base. Agitate until the drug is uniformly mixed in. Coat the formulation onto a release liner at a controlled thickness. Pass through an oven to drive off volatile solvents. Join the dried product to a backing material. Cut into individual patches and seal inside packages along with a desiccant pouch.

### Example 13

Dissolve ethylene-vinyl acetate (EVA) copolymer into an organic solvent. Add an ester conjugate of (2R,6R)-HNK free base, glycerol monolaurate, and a fatty acid ester co-solvent such as dodecyl acetate. Blend the mixture, cool, and press the wet blend into a 5 mm thick film. Laminate this drug reservoir film onto a spun-laced polyester backing layer. Apply a contact adhesive layer on the drug reservoir film, and then laminate a release liner to the contact adhesive layer. Cut into individual patches of appropriate size and insert into vacuum-sealed pouches.

### Example 14

Make a drug-adhesive emulsion of (2R,6R)-HNK free base, a styrene-isoprene-styrene block copolymer, polyisobutylene (high molecular weight), polyisobutylene (low molecular weight), alicyclic saturated hydrocarbon resin, and liquid paraffin. Apply the emulsion mixture onto a backing layer. Dry the adhesive wet blend to constant weight. On the other side, laminate with a release liner. Cut into individual patches and seal inside pouches.

### Example 15

Make a drug-adhesive emulsion of an ester conjugate of (2R,6R)-HNK free base, an acrylic pressure-sensitive adhesive (such as PLASTOID B, EUDRAGIT E, or DURO-TAK 387-2516), and a polyisobutylene pressure-sensitive adhesive (such as OPPANOL B10 or B100 made by BASF). Optionally, further add a silicone adhesive, such as BIO-PSA 4301. Stir the drug-adhesive emulsion with a blade stirrer. Apply the mixture onto a fluoropolymer-coated polyester film and dry for 10 min. at room temperature, and then another 20 min. at 60° C. in a cabinet drier. Cover the drug-containing layer with a light-opaque metal foil backing layer.

### Example 16

Make a drug-adhesive emulsion of a lysine conjugate of (2R,6R)-HNK free base, a styrene-isoprene-styrene block copolymer, an aliphatic hydrocarbon resin, oleic acid, and isopropyl myristate. Apply the wet blend onto a thin poly(ethylene-vinyl acetate) foam film. After drying, attach a siliconized polyester film (75 μm) onto the other side as a release liner. In alternate formulations, further add a poly(ethylene-vinyl acetate) adhesive. Optional substitute ingredients are a thermoplastic modified terpenic resin, oleic acid, or isostearyl isostearate.

### Example 17

Make the adhesive formulation by mixing together KRATON D1111KT (a styrene-isoprene-styrene block copolymer) and ARKON P-100 (hydrogenated hydrocarbon resin). Add (2R,6R)-HNK free base into the adhesive formulation and agitate until the drug is mixed therein. Spread the mixture onto a release liner and pass through an oven to evaporate the volatile solvents. Laminate a backing layer onto the dried drug-in-adhesive layer. Cut the laminate into individual patches and place into individual pouches. Optional additional ingredients in the adhesive formulation are polyisobutylene adhesive, GELVA 3087 adhesive, or DURO-TAK 87-900A adhesive (non-functional acrylic adhesive).

### Example 18 (Heat-Melt)

Mix styrene-isoprene-styrene block copolymer (provided as a powder), polyisobutylene, terpene resin, oleic acid (or alternatively, isostearic acid), and liquid paraffin into 1,3-butanediol solvent (or alternatively, dipropylene glycol solvent). Dissolve the mixture under heat. Separately, mix a lysine conjugate of (2R,6R)-HNK free base and oleic acid (or alternatively, isostearic acid) into 1,3-butanediol solvent (or alternatively, dipropylene glycol) under heat and add to the adhesive mixture to obtain a homogeneous plaster. Apply the wet blend onto a silicon-treated polyester film. Paste a polyester woven fabric onto the plaster and cool. Cut into individual patches.

### Example 19 (Heat-Melt)

Charge an extruder equipment with EUDRAGIT RS 100 granules (copolymer of ethyl acrylate and methyl methacrylate with low content of trimethyl ammonium ethyl methacrylate chloride). Heat the mixture to melt the adhesive. Add (2R,6R)-HNK free base to the melted adhesive and mix. Extrude the mixture into a thin sheet from a slit nozzle onto a siliconized polyester film as the release liner. Press the laminate by passing through cooling rollers. Cover the two-layer laminate with a backing layer to result in a three-layer laminate. Cut into individual patches.

### Example 20 (Heat-Melt)

Mix together styrene-isoprene-styrene block copolymer in powder form (such as KRATON D-1111), butyl rubber, hydrogenated hydrocarbon resin (such as ARKON P-100), and zinc oxide. Melt the mass under heat and add (2R,6R)-HNK free base to the mixture. Cast the mass onto a nonwoven fabric as the backing layer and cool. Apply a terephthalate film onto the other side as a release liner. Cut into individual patches.

### Example 21 (Matrix-Diffusion Patch)

Mix an acrylic copolymer into water. Separately, mix purified gelatin, polyvinyl alcohol, and an ester conjugate of (2R,6R)-HNK hydrochloride into water and stir at 60° C. for about 3 minutes. Combine the two mixtures and add glycerin. Blend together and spread onto a backing layer of nonwoven fabric to form a drug reservoir. Coat the edges of the backing layer with an acrylic adhesive to form an adhesive rim. Lay a release liner over the drug matrix and the adhesive rim.

### Example 22 (Matrix-Diffusion Patch)

Charge an extruder with EUDRAGIT RS 100 granules (copolymer of ethyl acrylate and methyl methacrylate with low content of trimethyl ammonioethyl methacrylate chloride), tributyl citrate, and (2R,6R)-HNK free base. Heat to melt the mixture. Extrude the heat-melted mixture onto a 100 μm thickness siliconized polyester film serving as the release liner for the patch. Feed the two-layer laminate through cooling rollers and then cover with a 20 μm thickness polyester film serving as the separating film between the drug matrix and the adhesive layer. From this three-layer laminate, stamp out discs of the drug matrices, cutting through the separating film and the drug-adhesive layer, but not the release liner. This results in forming an array of individual drug matrix discs on the release liner. On this array of stamped drug matrix discs, lay a double-sided self-adhesive tape film made of crosslinked acrylate copolymer. On top of this self-adhesive film, lay a polyurethane film which will serve as the backing layer for the patch. Cut this laminated assembly into individual patches having a configuration similar to the patch shown in FIGS. 5A and 5B.

### Example 23 (Multiple Drug Layers)

Make the outer drug layer (adhering to skin) by mixing 100 parts of a styrene-butadiene-styrene block copolymer adhesive, 175 parts of a glycerol ester of partially hydrogenated rosin, and 50 parts of rosin into n-heptane solvent. To this mixture, add (2R,6R)-HNK free base and acetic anhydride. Agitate to homogenize and then spread onto a siliconized polyester film at a thickness of 100 μm, which will serve as the release liner for the patch. Dry the moist film for 20 minutes at 50° C. Make the inner drug layer by mixing together 100 parts of a styrene-butadiene-styrene block copolymer adhesive and 20 parts dioctyl cyclohexane into n-heptane solvent. Add to this mixture, more of the (2R,6R)-HNK free base and acetic anhydride. Agitate to homogenize the mixture. Spread the wet blend as a 300 μm layer onto a siliconized polyester film, which will be detached during the manufacturing process. Dry for 20 minutes at 50° C.

Make the adhesive interlayer by mixing together 100 parts of a styrene-butadiene-styrene block copolymer adhesive, 175 parts of a glycerol ester of partially hydrogenated rosin, and 50 parts of dioctyl cyclohexane into n-heptane solvent. Spread the mixture as a 100 μm thickness layer onto a siliconized polyester film, which will be detached during the manufacturing process. Dry for 20 minutes at 50° C. Assemble the patch by laminating the inner drug layer onto the outer drug layer and then remove the siliconized polyester film attached to the inner drug layer. Laminate the adhesive interlayer onto the inner drug layer and then remove the siliconized polyester film attached to the adhesive interlayer. Laminate a 12 μm thickness polyester film, which will serve as the backing layer, onto the adhesive interlayer. Cut this finished laminate into individual patches.

In vitro or in vivo evaluations of the above transdermal patch examples can be performed according to the guidance given in Heather Benson & Adam Watkinson, supra; A. Arunachalam et al, supra; Dipen Patel et al, supra; Richa Sachan et al, supra; Ajay Sharma et al, “Transdermal Drug Delivery System: A Review” (2013) International Journal of Research in Pharmaceutical and Biomedical Sciences, vol. 4(1):286-292; U.S. Pat. No. 9,205,061 (Richard Hamlin et al); U.S. Pat. No. 7,608,282 (Peter Altenschöpfer et al); U.S. Pat. No. 8,524,273 (Satoshi Amano et al); U.S. Pat. No. 5,494,680 (Timothy Peterson); U.S. Pat. No. 9,314,470 (Prashant Patel et al); U.S. Pat. No. 9,314,527 (Jeremy Cottrell et al.); U.S. Pat. No. 9,155,712 (David Kanios et al.); U.S. Pat. No. 9,265,752 (Shuming Wang et al); U.S. Pat. No. 6,264,980 (Thomas Hille); U.S. Pat. No. RE 41,408 (Robert Reder et al); U.S. Pat. No. 6,953,590 (Yukino Owaki et al); U.S. Pat. No. 5,698,217 (Shari L. Wilking); U.S. Pat. No. 9,283,174 (Tatsuya Mori et al); U.S. Pat. No. 6,555,129 (Christoph Arth et al); U.S. Pat. No. 9,168,232 (Naohisa Kawamura et al); U.S. Pat. No. 9,173,855 (Hiroko Udagawa et al); and U.S. Patent Application Publications No. 2014/0052081 (Kuo-Hua Yang et al); No. 2003/0026829 (Subramanian Venkatraman); No. 2008/0254073 (Sang Cheol Chi); No. 2016/0113908 (Abhijit Deshmukh et al); No. 2015/0250738 (Su II Yum et al); No. 2015/0246007 (Robert Gale et al); No. 2016/0051489 (Richard Hamlin et al); No. 2016/0030362 (Jun Liao et al); No. 2015/0283097 (Kensuke Murata); No. 2016/0113908 (AM Deshmukh et al); No. 2016/0051489 (Richard Hamlin et al.); No. 2015/0246006 (Kristin Jackson et al); all the preceding are incorporated by reference herein.

### 15. Conclusion

The foregoing description and examples have been set forth merely to illustrate my invention and are not intended to be limiting. Each of the disclosed aspects and embodiments of my invention may be considered individually or in combination with other aspects, embodiments, and variations of my invention. In addition, unless otherwise specified, the steps of the methods of my invention are not confined to any particular order of performance. Modifications of the disclosed embodiments incorporating the spirit and substance of my invention may occur to persons skilled in the art, and such modifications are within the scope of my invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A transdermal patch comprising:
a backing layer;
a release liner;
between the backing layer and the release liner, a drug-containing layer comprising 10-250 mg of an active agent that is (2R,6R)-hydroxynorketamine or a conjugate thereof.
